(19) United States
(12) Patent Application Publication
(10) Pub. No.: US 2024/0042230 A1
(43) Pub. Date: Feb. 8, 2024

(54) STAND-ALONE APPLIANCE FOR VIOLET LIGHT DELIVERY TO PREVENT OR SLOW THE PROGRESSION OF MYOPIA

(71) Applicant: TWENTY TWENTY THERAPEUTICS LLC, South San Francisco, CA (US)

(72) Inventors: Fred Lee, South San Francisco, CA (US); Dennis Gorshteyn, South San Francisco, CA (US); Supriyo Sinha, Menlo, CA (US)

(21) Appl. No.: 18/229,550

(22) Filed: Aug. 2, 2023

Related U.S. Application Data
(60) Provisional application No. 63/394,385, filed on Aug. 2, 2022.

(51) Int. Cl. A61N 5/06

(52) U.S. Cl. CPC A61N 5/0613; A61N 2005/0663; A61N 2005/0626

(57) ABSTRACT

A stand-alone appliance can detect a user's face/eyes and provide violet light to the user. The appliance can include a fixture mounted on a platform and at least one motor to move the platform. A directed light source, coupled to a processor, can emit a light signal directed to an adjustable focal point. A camera, coupled to the processor, can detect the user and provide an image of the user's face to the processor. The processor can signal the motor to orient the platform to bring an eye into a center of a frame and adjust the focal point. A distance sensor, coupled to the processor, can estimate a distance between the fixture and the eye to ensure that an appropriate optical energy density is applied to the user. The camera, the directed light source, and/or the distance sensor can be embedded in the fixture.

### CROSS-REFERENCE TO RELATED APPLICATIONS

This application claims the benefit of U.S. Provisional Application Ser. No. 63/394,385, filed 2 Aug. 2022, entitled “STAND-ALONE APPLIANCE FOR VIOLET LIGHT DELIVERY TO PREVENT OR SLOW THE PROGRESSION OF MYOPIA”. The entirety of this provisional application is incorporated by reference for all purposes.

### TECHNICAL FIELD

The present disclosure relates generally to preventing or slowing the progression of myopia and, more specifically, to systems and methods employing a stand-alone appliance that delivers VL to a user's eye(s) to prevent or slow the progression of myopia.

### BACKGROUND

Myopia, also known as nearsightedness, is a common refractive disorder of the eye that is prevalent around the world and the prevalence is increasing. In fact, it is projected that 50% of the world population will be affected by myopia by 2050. A person suffering from myopia can see close objects clearly, but distant objects appear to be blurred. Myopia tends to develop and progress the most rapidly during childhood and adolescence but can occur or worsen anytime throughout the person's life. As the person increases near vision tasks, like using computers, smartphones, tablets, etc., and/or spends more time indoors (where indoor lighting rarely contains light with wavelengths under 400 nm), the risk for developing or worsening myopia is thought to increase. One method for mitigating the risk of myopia or slowing the progression of myopia is for the person to spend a certain amount of time outdoors (e.g., in sunlight that includes wavelengths less than 400 nm). However, for many people in the developed world, regular time spent outdoors in unobstructed sunlight for the amounts of time needed to slow the occurrence of myopia is becoming harder to achieve (e.g., due to school requirements, climate change, increased computer and phone use, etc.). In people who cannot or will not spend the appropriate amount of time outdoors, other methods are needed to increase the amount of light with wavelengths below 400 nm (such as violet light) that they are exposed to in order to slow or prevent progression of myopia.

### Summary

A stand-alone appliance can be used to deliver violet light to a user to slow or prevent the progression of myopia. The stand-alone appliance can identify the user and deliver a predefined amount of violet light to the user from a violet light source aimed toward the user's eyes while operating at a low power. The present disclosure relates to systems and methods employing the stand-alone appliance to prevent or slow the progression of myopia.

In an aspect, the present disclosure includes a system to prevent or slow the progression of myopia. The system can include a fixture mounted on a platform and at least one motor configured to move the platform. The system can also include a directed light source, coupled to a processor, that can to emit a light signal (containing violet light) directed to an adjustable focal point; a camera, coupled to the processor, that can detect a presence of a user and provide an image of the user's face to the processor, wherein the processor signals the at least one motor to orient the platform to bring an eye into a center of a frame of and begin to adjust the adjustable focal point of the directed light source; and a distance sensor, coupled to the processor, that can estimate a distance between the fixture and the eye to ensure that an appropriate optical energy density is applied to the user. It should be noted that at least one of the camera, the directed light source, and the distance sensor can be embedded within the fixture.

In another aspect, the present disclosure includes a method for preventing or slowing the progression of myopia. Steps of the method can be performed by a system including a processor. The steps of the method include recognizing the presence of a user via at least one camera in communication with the processor; identifying the user from a stored group of one or more users of the system; and providing a directed therapeutic light treatment (e.g., violet light) to at least one eye of the user via a directed light source. At least one of the directed light source and the at least one camera are moveable by at least one motor in communication with the processor.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 shows a diagram of a system that can employ a stand-alone appliance that delivers violet light to a user to prevent or slow the progression of myopia;

FIG. 2 shows example components of the fixture of FIG. 1;

FIGS. 3 and 4 show example implementations of the system of FIG. 1;

FIG. 5 shows an example of firmware that can be used to operate the system of FIG. 4;

FIG. 6 is a process flow diagram illustrating a method for employing a stand-alone appliance that delivers violet light to a user to prevent or slow the progression of myopia;

FIG. 7 is a process flow diagram illustrating a method for detecting a user;

FIG. 8 is a process flow diagram illustrating a method for delivering VL to a user's eye;

FIG. 9 is a process flow diagram illustrating a method for regulating an amount of VL treatment delivered to a user; and

FIG. 10 is a process flow diagram illustrating a method for detecting eyewear of a user designed to block light.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “myopia”, also referred to as “nearsightedness”, can refer to a common vision condition in which objects that are near are seen clearly, but objects farther away are blurry.

As used herein, the term “violet light”, also referred to as “VL”, can refer to light at the short wavelength end of the visible spectrum (with a shorter wavelength than blue light) and may include ultraviolet light. VL can be administered to a user as light signal used as a treatment (also referred to as a light treatment) for a user to prevent or slow progression of myopia. As an example, violet light can have a wavelength between 310 nm and 450. As another example, violet light can have a wavelength between 360 nm and 400 nm.

As used herein, the term “stand-alone appliance” can refer to a device that is substantially free-standing and not integrated into or attached to a device with another purpose.

As used herein, the term “fixture” a supporting device that can be mounted on a platform and can include one or more components (e.g., the one or more components can be embedded within or attached to the fixture). The components can include a camera, a directed light source, and/or a distance sensor, for example.

As used herein, the term “motor” can refer to hardware that imparts motion. For example, a motor can be used to move the platform in at least one degree of freedom (a direction of motion that has the freedom to vary). For example, the motion can be left/right motion, up/down motion, rotation, etc., caused by a servo motor.

As used herein, the term “ambient light” can refer to any light that illuminates the environment surrounding a user. As an example, ambient light may be natural light (e.g., sunlight) or artificial light (e.g., from a lamp or overhead light).

As used herein, the term “user”, which may also be referred to as a “subject”, a “patient”, or the like, can refer to a human being of any age employing the stand-alone appliance to deliver VL to at least one eye with the intent of preventing or slowing the progression of myopia.

### II. Overview

The present disclosure relates generally to preventing or slowing the progression of myopia with violet light. Myopia is a common disorder of the eye where a person can see close objects clearly, but distant objects appear to be blurred. Myopia is already quite common, and its prevalence is steadily increasing around the world as people engage in more near vision tasks and/or spend more time indoors. It is thought that the development and/or progression of myopia can be mitigated or slowed by exposure to unobstructed sunlight for a certain amount of time daily and or weekly. However, for many people in the developed world, regular time spent outdoors in unobstructed sunlight for the amounts of time needed to slow the occurrence of myopia is becoming harder to achieve (e.g., due to school requirements, climate change, increased computer and phone use, etc.). It has been hypothesized that outdoor light and indoor light differ in the fact that outdoor light naturally includes violet light wavelengths while indoor light generally excludes such wavelengths. Accordingly, violet light can be delivered into the person's eye with the hope of achieving the same results as spending the appropriate time outdoors.

As described herein, a stand-alone appliance (e.g., sized on the order of a standard 12 ounce beverage can) can be used to deliver violet light to a user to slow or prevent the progression of myopia. The stand-alone appliance can be used by anyone who cannot spend an appropriate amount of time outdoors to prevent or slow the progression of myopia. However, it is hypothesized that children, teenagers, and college students have a particular need for such a violet light treatment and can receive the greatest benefit therefrom. The stand-alone appliance can include a camera imaging system for face/eye detection and a motorized violet light source that tracks the detected face/eyes and aims the light into at least one of the detected eyes, while not exposing the rest of the user to the light. The stand-alone device can operate at a low power (e.g., less than 0.31 W/cm 2). In some instances, the stand-alone device can identify a user from a group of users, determine how much violet light the user has already received, and ensure that the appropriately predefined amount of violet light is delivered to the user. Accordingly, the present disclosure relates to systems and methods employing the stand-alone appliance to prevent the development or slow the progression of myopia.

### III. Systems

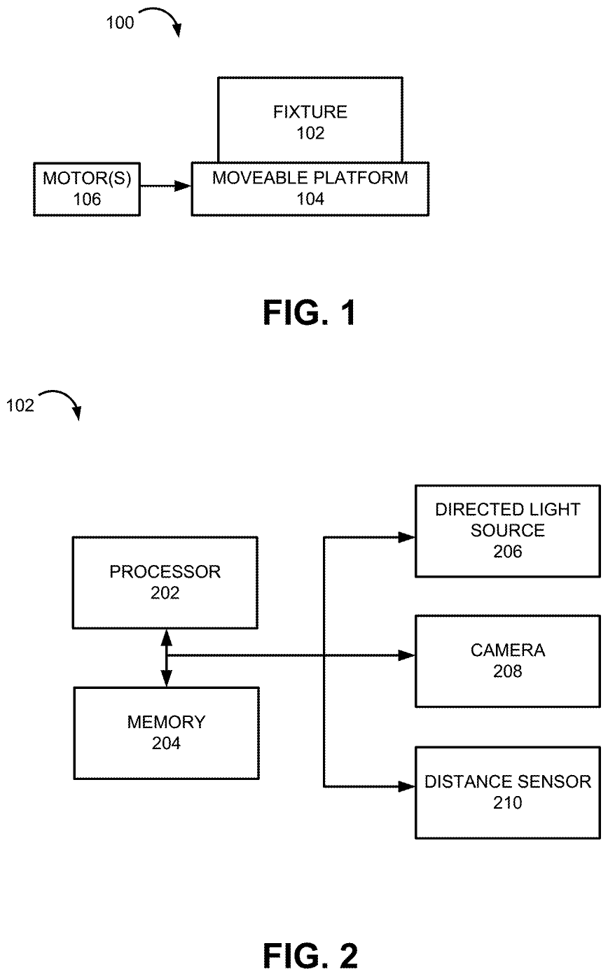

One aspect of the present disclosure includes a system 100 (FIG. 1) that can employ a stand-alone appliance to deliver violet light (VL). As an example, the stand-alone appliance can configure and/or deliver a VL therapy to a user's eye. The stand-alone appliance can be substantially free-standing and not integrated into or attached to a device with another purpose. As one example, the stand-alone appliance can be sized on the order of a standard 12 ounce beverage can. In some instances, the stand-alone appliance can be shaped like a standard 12 ounce beverage can. However, it should be understood that the stand-alone appliance can be any shape and/or size and the standard 12 ounce beverage can is only an example of size and shape. The stand-alone appliance can be configured to deliver VL therapy to one or more eyes of a user using one or more directed beams of VL. It should be understood that VL generally includes light at the short wavelength end of the visible spectrum (e.g., wavelengths less than those of blue light) and may include ultraviolet light. As an example, VL can have a wavelength between 310 nm and 450 nm. As another example, VL can have a wavelength between 360 nm and 400 nm.

The VL therapy can be delivered by the stand-alone appliance to one or more of a user's eyes to prevent or slow the progression of myopia (also referred to as nearsightedness) in one or more of the user's eyes. Myopia can develop or worsen for the user at any age, especially due to increased near vision tasks, such as using computers, smartphones, tablets, etc., spending more time indoors, or the like. Therefore, one or more eyes of any user (at any age) may benefit from the delivery of VL. However, because myopia tends to develop and progress the most rapidly during childhood and adolescence, one or more eyes of users under age 25 may see more benefit from the delivery of VL. The stand-alone appliance can be positioned near a place a user will spend a certain amount of time without moving excessively (e.g., at a homework spot, near a computer, near a TV, etc.). For example, the stand alone appliance can be portable such that a user can move the stand-alone appliance from location to location as needed.

The stand-alone appliance can apply the VL therapy as a directed light beam towards one or more eyes of the user, while minimizing the violet light that touches other parts of the user (e.g., face, hands, neck, etc.). For example, the VL therapy can be applied to one eye at a time (e.g., completing a treatment for one eye then moving to the other eye), the VL therapy can be applied simultaneously (e.g., with two light beams), or the VL therapy can be applied concurrently (e.g., switch from eye to eye until both eyes have received a full treatment dose). The stand-alone appliance can be configured for use by multiple users and can be configured to detect what user is being treated at a time by facial recognition. The stand-alone appliance can keep profiles of each user that can include facial and identity data, age, pertinent health related data, and dosage amounts and schedules (including if a partial dosage was applied within a given time period and needs to be completed). The stand-alone appliance can also be configured to detect the eye of the user identified so that the stand-alone appliance can properly direct the light beam towards the eye (e.g., pupil) of the user for best results. The stand-alone appliance can also detect if the user is wearing a violet light blocking lens (e.g., glasses or contacts comprising a coating or material that filters violet light) and require the user remove said lenses before the VL therapy can be applied.

In its simplest form, the system 100 can include a fixture 102, a moveable platform 104, and one or more motors (motor(s) 106). The fixture 102 can be positioned on the moveable platform 104. As an example, the fixture 102 can be mounted on the moveable platform 104 by mechanical means (e.g., welded, extruded, 3D printed, screwed, adhesive, etc.), may be part of the moveable platform (e.g., formed together), etc. The one or more motors (motor(s) 106) can be configured to move the moveable platform 104 and thereby the fixture 102 mounted on the moveable platform with at least one degree of freedom. For example, the one or more motors (motor(s) 106) can be configured to translate the moveable platform 104 and/or rotate the moveable platform. In one example, each of the one or more motors can cause the moveable platform 104 to move in at least one of a left/right motion, an up/down motion, or a rotational motion, or the like. In another example, the at least one motor (motor(s) 106) can move the moveable platform 104 left/right and tilt the moveable platform up and down. The one or more motors can be at least one of a servo motor, a linear motor, a servo motor, an AC motor, a DC motor, a direct drive motor, or the like. The one or more motors (motor(s) 106) can be positioned external to or inside of the stand-alone appliance.

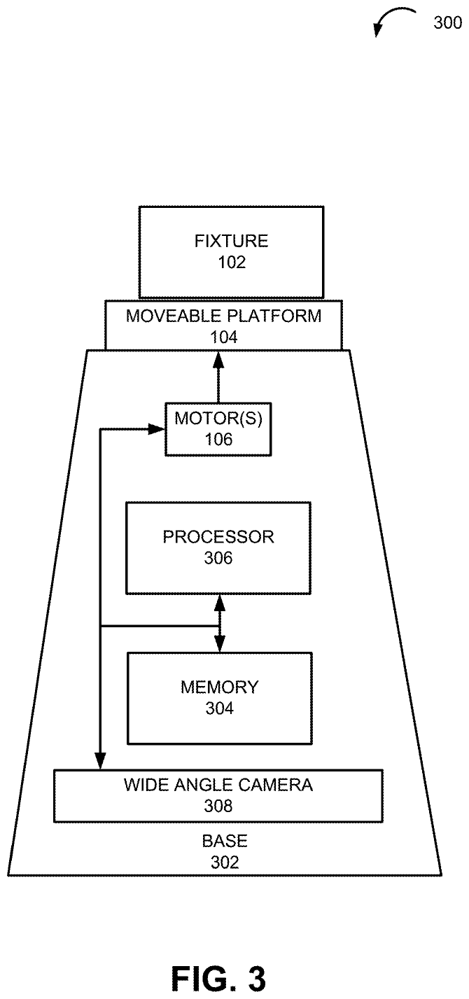

The fixture 102 can have one or more components attached thereto and/or housed therein for the purposes of generating VL therapy to be directed to one or more eyes of a user. The fixture 102 can be, for example, a housing and/or a backbone-like structure. The fixture 102 can be at least partially one or more of a, for example, a polymer or a metal material. As shown in FIG. 2, the fixture 102 can include components such as a processor 202, a memory 204, and a plurality of components, including but not limited to, a directed light source 206, a camera 208, and a distance sensor 210. Other components may also be attached to and/or housed within the fixture 102. In some instances, the processor 202 can perform the actions reflected in the instructions stored in the memory 204. For example, the processor 202 can be a microprocessor that can perform actions related to the memory 204 and the processor 202. The fixture 102 can act as a support or housing for the memory 204, the processor 202, and the one or more components. In fact, at least one of the one or more components (e.g., directed light source 206, camera 208, distance sensor 210, etc.) can be embedded within the fixture 102 or attached thereto. However, all of the components can be set on the moveable platform 104, whether embedded in the fixture 102 or not embedded within the fixture 102.

As shown in FIG. 2, the directed light source 206, the camera 208, and the distance sensor 210 can be in electrical communication with the processor 202 and the memory 204 to provide directed VL therapy to one or more eyes of a user. Providing directed VL therapy to one or more eyes of a user can include at least generating VL and shining VL into one or more of the user's eyes for a time. The directed light source 206 can emit the VL and can be moveable to direct the VL into at least one of the user's eyes. The camera 208 can detect a presence of the user and/or locate at least one of the user's eyes. The distance sensor 210 can determine how far away the user is from the stand-alone appliance and guide the directed light source 206 to direct the VL to the at least one eye of the user in a focused manner. A directed light source 206, camera 208, and distance sensor 210 are shown in FIG. 2, but alternative and/or additional components may be utilized for providing directed VL therapy to one or more eyes of user and/or any other actions described herein. Each of the components can be coupled to the processor 202 (and, in some instances, the memory 204) with a wired or wireless connection to facilitate communication of data and instructions.

The directed light source 206 can emit a light signal directed to an adjustable focal point. The directed light source 206 can include one or more light sources (e.g., light bulbs, LEDs, OLEDs, PLEDs, AMOLEDs, lasers, etc.). The directed light source 206 may include one or more optical components (e.g., lenses, mirrors, etc.) to focus the light generated from an unfocused light source and/or to adjust the focal point of the light signal admitted from either an unfocused light source or a focused light source. The light signal can include the VL of at least one wavelength to be delivered as a treatment to the user. Accordingly, the directed light source 206 can include at least one VL source for treatment purposes. However, to aid the distance sensor 210, the directed light source 206 can also include one or more red light sources for measurement purposes. The directed light source 206 can include one or more light sources for emitting light of different wavelengths, for example, the directed light source can include at least one of a violet light source, an ultraviolet light source, a red light source, and an infrared light source, or the like. In one example, the directed light source 206 can include a violet light source and an infrared light source. In another example, the directed light source 206 can include a filter that can determine the wavelengths of light emitted. As an example, the violet and ultraviolet light can be used for treatment, while the red and infrared light can be used for measurements.

The camera 208 can be electrically coupled to the processor 202 (e.g., wired or wireless connection) and can detect a presence of a user (e.g., by using image capture at a predetermined frame rate and instructions on the processor to compare each frame for frame to frame changes that indicate the presence of a moving object/person who may be a user and to determine based on stored information if the moving object is or is not a person and is or is not a user of the system) and provide an image of the user's face (e.g., once the moving object/person is detected) to the processor 202. In response to receiving an image of the user's face the processor 202 can then signal the at least one motor (motor(s) 106 in FIG. 1) to orient the platform (movable platform 104) to bring an eye of the user into a center of a frame of the camera 208 and to begin to adjust the adjustable focal point of the directed light source 206 so that the focal point matches the location of the eye. The distance sensor 210 can be electrically coupled to the processor 202 (e.g., wired or wireless connection) and can estimate a distance between the fixture 102 and the eye of the user to ensure that an appropriate optical energy density is applied to the eye. The optical energy density can also be varied based on ambient light, an amount of VL already received by the user, or the like. The distance between each of the user's eyes also can be estimated. The distance sensor 210 can include an ultrasound emitter, a red light emitter, an infrared emitter, or the like. As noted, the ultrasound emitter, the red light emitter, the infrared emitter, or the like, can be within the directed light source 206. The distance sensor 210 can estimate the distance between the fixture 102 and the eye of the user based on reflectance of a light beam off an object (e.g., the eye of the user), for example an infrared light beam. The reflectance can be detected by the camera 208 in some instances.

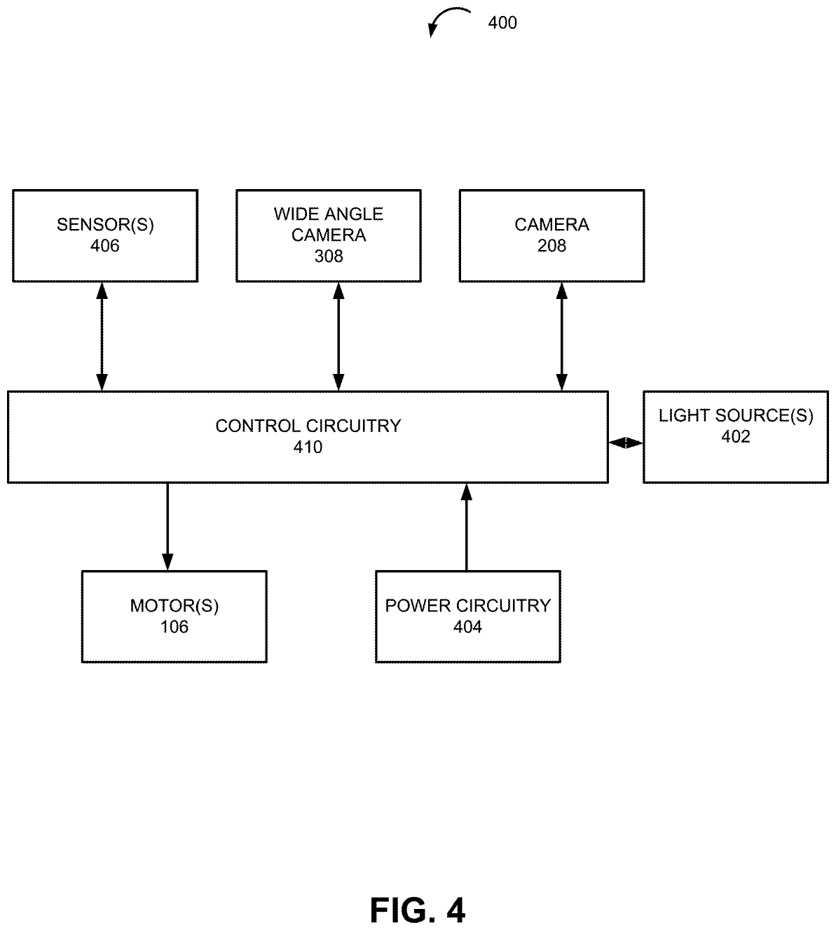

An example stand-alone appliance 300 is shown in FIG. 3. The example stand-alone appliance 300 includes a base 302 attached to the movable platform 104. The base 302 can house processor 306, memory 304, and, in some instances, the at least one motor (motor(s) 106), which can work as described above with respect FIG. 2 (with the processor 202 and the memory 204 housed in the fixture 102). The processor 306 and/or the memory 304 can also be separate from the base 302 (e.g., can be combined with the processor 202 and/or the memory 204 housed in the fixture). In some instances the fixture 102 and the base 302 can both include a processor 202 and 306 and a memory 204 and 304, that can either share or exclusively communicate with components of the fixture 102 and/or base 302. In other instances, the stand-alone appliance can include only one processor and memory, or two processors 202 and 306 and a shared memory. The base 302 can also include at least a second camera (wide angle camera 308) and an ambient light detector (not shown in FIG. 3), both of which can be electrically coupled with the processor 306 (and, in some instances, the memory 304). The at least the second camera can detect a user. For example, the at least the second camera can detect if a potential user (e.g., a human) appears within a frame of reference of the at least the second camera. The at least the second camera can be a wide angled camera 308, which has a wider field of view than a traditional camera. The ambient light detector can detect an illuminance of ambient light in the stand-alone appliance's surrounding and can adjust (within predefined safety thresholds) an exposure time and/or an intensity of the light signal emitted by the directed light source such that a user receives an appropriate amount of VL therapy. The ambient light detector can include, for example, an additional camera and/or processor/processing capability. In other instances, the ambient light detector can at least partially include the wide angled camera 308 and the processor 306. The base 302 can also include at least one of: a power source and/or a charging port, a user interface, a display, a speaker, and circuitry.

The processor 306 can execute instructions stored in the memory 304 (e.g., a non-transitory memory) to recognize the presence of a user (e.g., using the wide angled camera 308 to detect for a human) and recognize the identity of the user (e.g., using camera 208 of fixture 102 to capture image(s) of the user's face) from a stored group of one or more users of the system (with which the captured image(s) of the user's face are compared). For example, the stored group of one or more users of the system can be stored in the memory 304. The stored group of one or more users of the system can be input, for example by each user utilizing a user interface and following instructions for creating a facial recognition profile. The processor 306 can identify the user based on the image of the user's face detected by the camera 208 of fixture 102. After the processor 306 has recognized the presence and identity of the user, the processor can then provide a directed therapeutic light treatment to the at least one eye of the user, via the directed light source 206 of fixture 102. The processor 306 can determine a treatment length (and/or power) for the user based at least one of: a user profile, an amount of ambient light detected by the ambient light detector, and an amount of at least one type of light received by the user that day stored in the non-transitory memory 304 (e.g., light from a previous treatment session, light detected by a sensor worn by the user during daily activities that is in wireless communication with the memory 304, etc.), or the like.

FIG. 4 shows a block diagram 400 of the communication relationships (wired and/or wireless) between the components of the stand-alone appliance for providing direct VL therapy with control circuitry 410. The control circuitry 410, which can include processor(s) and memory(s), can receive power from the power circuitry 404. The power circuitry 404 can include a power source, such as one or more batteries, one or more rechargeable batteries, one or more panels for a renewable energy source (e.g., solar) and/or an external plug for AC and/or DC power. When the stand-alone appliance is powered, the control circuitry 410 can send instructions/commands to, and receive data from, the other components of the stand-alone appliance. For example, the control circuitry 410 can send instructions to the wide angle camera 308 to detect at a given rate for the presence of a user and/or a human and can then receive the image data and determine if a person's (e.g., a user's) presence has been detected. Once a user's presence has been detected by the control circuitry 410, then the control circuitry can instruct the camera 208 to detect images of the person/user and send the image data to the control circuitry. The control circuitry 410 can the determine the identity of the user based on the image data from camera 208 based on a user image database. The control circuitry 410 can also send instructions to the motor(s) 106 to translate (e.g., right/left, up/down, etc.) and/or rotate to adjust directions so that the light source(s) 402 are aimed at the user's eye. Once the light source(s) 402 are aimed at the user's eye, the motor(s) 106 can stop moving the light source(s) 402 and the light source(s) can deliver a predefined dosage of VL to the user's eye. The motor(s) 106 can also move the light source(s) 402 as needed during delivery of the VL to maintain the VL therapy directed to at least one of the user's eyes if the user's moves (e.g., fidgets, shifts, tilts head, etc.).

The control circuitry 410 can also communicate with and control the sensor(s) 406. The sensor(s) 406 can include, but are not limited to, a distance sensor and an ambient light detector. The sensor(s) 406 can be distinct sensors or can be at least partially embodied with the wide angle camera 308, the camera 208, and the light source(s) 402. For example, the distance sensor can include an infrared light source the reflectance of which (e.g., off a part of a person) can be detected by one of the camera 208 and/or the wide angle camera 308, or another photodetector of the distance sensor itself. In another example, the ambient light detector can detect ambient violet light levels and/or if the user is wearing a violet or UV light reflecting pair of lenses (e.g., glasses or contact lenses or the like). To detect ambient violet light levels the sensor(s) 406 can include an additional photodetector and/or utilize the camera 208 and/or the wide angle camera 308. To detect if a user is wearing a violet or UV light reflecting pair of lenses the sensor(s) 406 can utilize a violet light source and measure reflectance near the eye utilizing an additional photodetector and/or utilize the camera 208 and/or the wide angle camera 308. The control circuitry 410 can instruct the sensor(s) 406, the wide angle camera 308, the camera 208, and/or the light source(s) 402 to complete at least the above described operations.

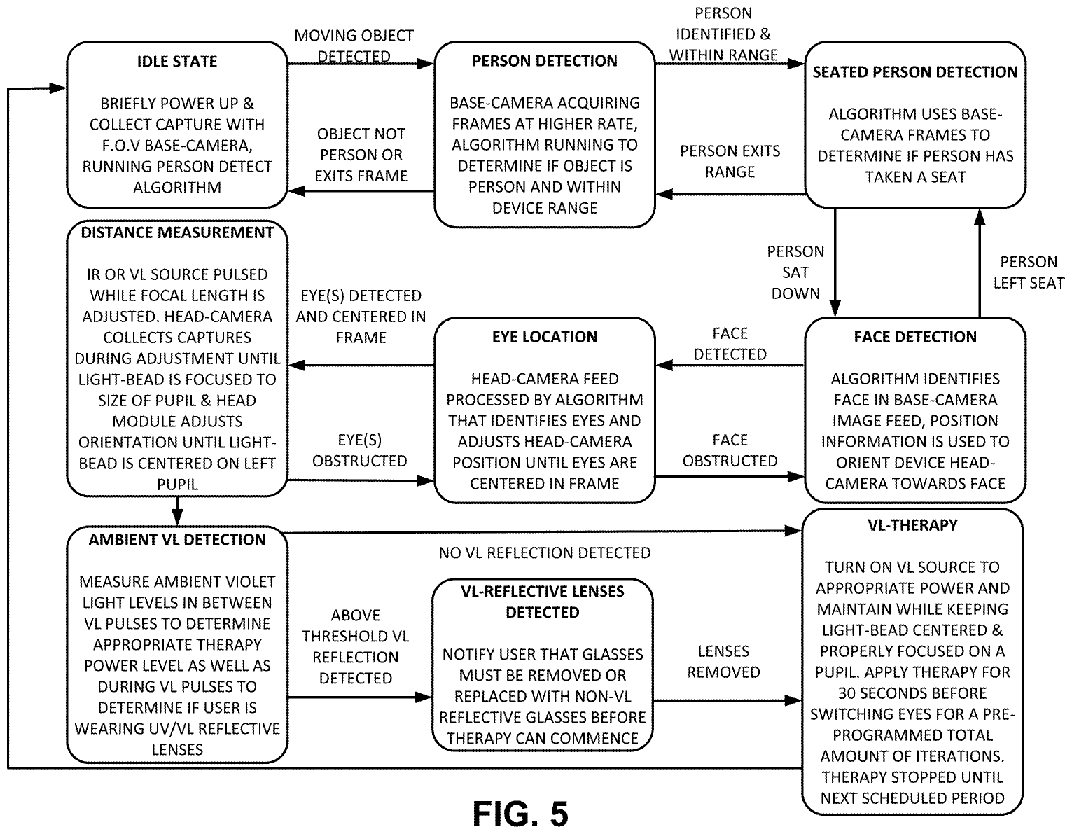

FIG. 5 shows an example flow chart of processes the stand alone appliance can execute to provide directed VL light therapy to a user. Not all processes shown are necessary for every provision of the directed therapeutic light and may be skipped. The stand-alone appliance can start in an Idle State where the stand-alone appliance can briefly power up (e.g., for a second, a few seconds, etc. every 10 seconds, 30 seconds, etc.). and collect image capture with a field of view camera in the camera in the base of the stand-alone appliance (in some instance the camera in the fixture may also be used) and the processor can run a person (or movement) detect algorithm to detect if a moving object, such as a person, who may be a user, is in the detection range of the stand-alone appliance. If the stand-alone appliance does not detect a moving object, then it remains in the Idle State. If the stand-alone appliance does detect a moving object, then the stand-alone appliance moves to a Person Detection state. In the Person Detection State the base camera (or in some instances the fixture camera) can acquire frames at a higher rate than during the Idle State, and the process can run an algorithm to determine if the object detected by the camera is a person and if that person is within the range of the stand-alone appliance (e.g., the device). If the moving object was not a person (e.g., an animal or inanimate object) or if the person exists the frame before the person detection can be complete, then the stand-alone appliance returns to the Idle State. If a person is detected and remains within range of the stand-alone appliance, then the stand-alone appliance moves to Seated Person Detection mode and with an algorithm, and the base camera (or in some instance the fixture camera) frames still being acquired, determines if the person has taken a seat. If the person has not taken a seat, and optionally exits range then the stand-alone appliance goes back to person detection mode.

If the person is determined to have sat down within range of the stand-alone appliance, then the stand-alone appliance enters Face Detection mode (e.g., to recognize if the person is a user). In Face Detection mode the stand-alone appliance runs an algorithm to identify the face in the frames recorded by the base camera (or in some instances the fixture camera) from the camera feed and position information is used to orient the stand-alone appliance's (e.g., device's) head camera towards the user's face. The user can be identified from a group of users in a database. If the face of the user cannot be detected, for example, the person left their seat, then the stand-alone appliance reverts back to Seated Person Detection mode. If the face is detected, and identified, then the stand-alone appliance enters an Eye Location Mode where the head camera (e.g., fixture camera) (or in some instances the base camera) feed is processed by an algorithm to identify the eyes of the user and that can adjust the position of the head camera (e.g., fixture camera) until the eyes (or at least one eye) is centered in the frame of the head camera. The adjustments can be done by one or more motors of the stand-alone appliance controlled by the algorithm. If the eyes (or eye) cannot be located (e.g., the face is obstructed by hair or a ball cap or the like) then the stand-alone appliance is returned to Face Detection mode. If the eyes (or eye) are detected and can be centered in the frame of the head camera (e.g., fixture camera), then the stand-alone appliance starts Distance Measurement mode. In Distance Measurement mode an infrared light or a violet light source of the stand-alone appliance can be pulsed while the focal length of the violet light source is adjusted. The head camera (e.g., fixture camera) (or in some instances the base camera) can collect capture images of the face of the user during the adjustment flashes until the processor can determine that the light-bead is focused to the size of the pupil and is focused on one of the pupils (e.g., the left or the right pupil, although left is shown). The head module (e.g., the fixture on the platform can be moved by the at least one motor).

When the Distance Measurement mode is completed, and the violet light source is focused at a pupil of the user then the stand-alone appliance can move into Ambient Violet Light (VL) Detection mode. In Ambient Violet Light Detection Mode, the stand-alone appliance can measure ambient violet light levels in between violet light pulses (or in some instances when no lights are pulsing) to determine an appropriate therapy power level (e.g., intensity) based on the ambient violet light. The Ambient Violet Light Detection Mode can also determine during the violet light pulses if the user is wearing UV and/or violet light reflective lenses (e.g., glasses or contacts) based on if a threshold of violet light reflection is exceeded in camera frames of the user during the Ambient Violet Light Detection mode. If no violet light reflection is detected, then the stand-alone appliance moves directly into Violet Light (VL)-Therapy mode. If the violet light reflection above a threshold is detected, then the stand-alone appliance detours to a Violet Light (VL)-Reflective Lenses Detected State. In the Violet Light (VL)-Reflective Lenses Detected State the stand-alone appliance notifies the user that the lenses (e.g., glasses or contacts) must be removed and/or replaced with non-violet light reflective lenses before therapy can commence. Such a notification can be audible, visual, and/or haptic and can be provided, for example, by a display or speaker associated with the stand-alone device. The stand-alone device can deactivate if the user does not remove said lenses within a certain number of notifications within a time period. If the user removes the lenses, then the Violet Light (VL)-Therapy mode starts. In Violet Light (VL)-Therapy mode the violet light source is turned on to an appropriate power (for the therapy) and maintained for the time period of the therapy while keeping the light bead (e.g., focal point) centered and properly focused on a pupil of the user. In one example, the therapy can be applied to one eye for 30 seconds and then switched to the other eye for another 30 seconds, for a preprogrammed total amount of iterations. When the total therapeutic dose has been delivered then the stand-alone appliance can stop the therapy until the next scheduled period for therapy (e.g., the next day). In one instance, if the therapy is interrupted, then the stand-alone appliance can record the therapy level (amount) received for that specific user in case the user comes back later in the same scheduled therapy period, such that the user only receives a single total therapeutic dose for the scheduled therapy period.

### IV. Methods

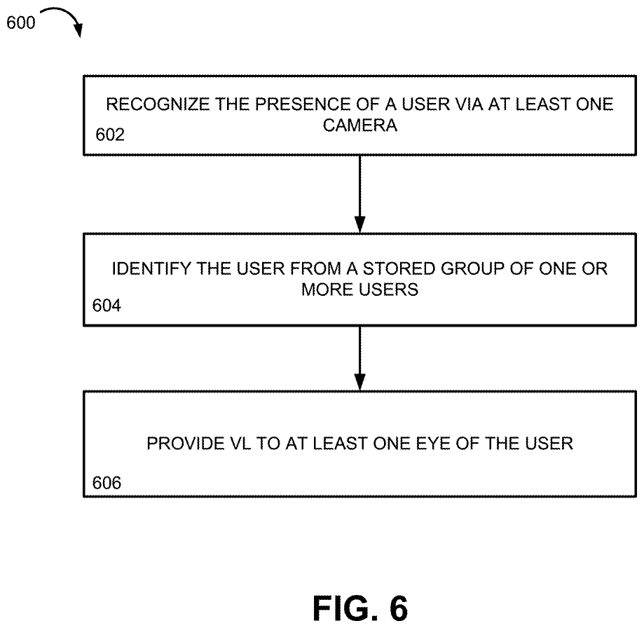

Another aspect of the present disclosure can include methods 600-1000 (FIGS. 6-10) for preventing or slowing the progression of myopia using a stand-alone appliance that can provide violet light treatment to a user. The methods 600-100 can be executed using the any of the systems and devices described above with respect to FIGS. 1-5.

The methods 600-1000 are illustrated as process flow diagrams with flowchart illustrations. For purposes of simplicity, the methods 600-1000 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 600-1000.

Referring now to FIG. 6, illustrated is a method 600 for employing a stand-alone appliance that delivers violet light to a user to prevent or slow the progression of myopia. At step 602, the presence of a user can be received via at least one camera in communication with a processor of the system (e.g., the stand-alone appliance). The presence of the user can be recognized by detected a moving object in the frame of the at least one camera and then determining if the moving object is or is not a human and detecting if the human is in range of the camera and/or the directed light source of the stand-alone appliance. At step 604, the user can be identified from a stored group of one or more users of the system. The system can access a database containing information about faces of the stored group of one or more users of the system (e.g., that the users have previously stored during a set-up process). The system can then perform facial recognition, by any given means, of the face of the detected user (e.g., the detected human) compared with the information about the faces of the stored group of one or more users of the system. The human can be identified as a specific user of the system if the face matches with the information about one of the faces of the stored group of one or more users of the system. At step 606, a directed therapeutic light treatment can be provided to at least one eye of the user via a directed light source of the system. At least one of the directed light source and the at least one camera are moveable by at least one motor in communication with the processor to complete the above steps. The directed light source can move to follow the movement of the at least one eye as the user moves. In some instances, the intensity and/or time of the directed VL therapy can be altered based on which user is identified (e.g., based on specific treatment protocols, sensor information, previous treatment during a given time period etc.).

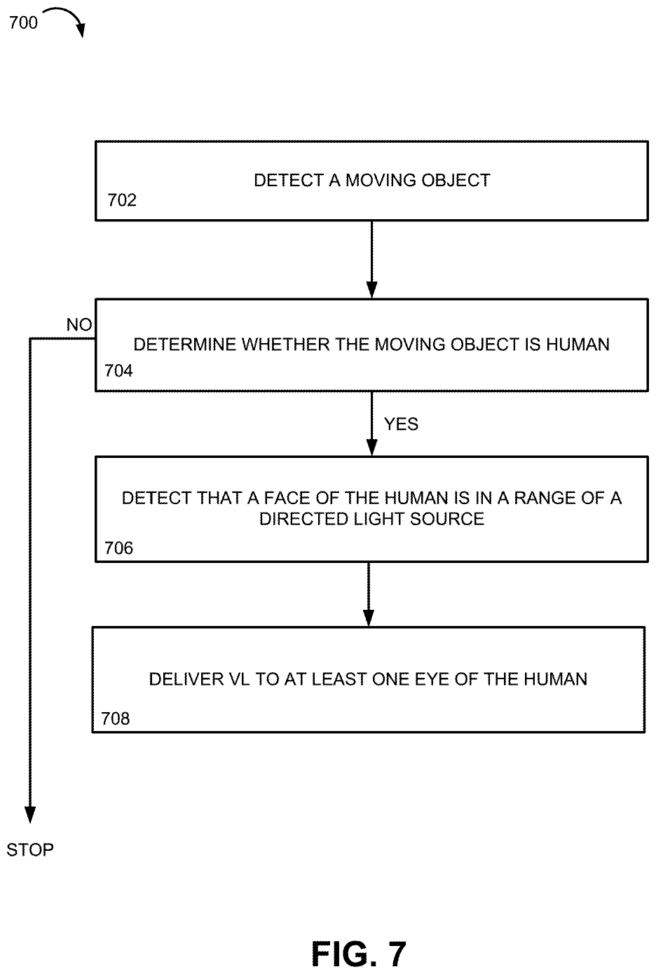

Referring now to FIG. 7, illustrated is a method 700 for detecting a user. At step 702, a moving object can be detected by the processor in communication with the at least one camera. The at least one camera can be recording at a given rate and the processor can detect movement based on the changes in the images recorded within a given time frame. At step 704, it can be determined that the moving object is a human. For example, the system can determine the moving object is a human based on a pattern recognition or another predetermined method utilizing the at least one camera and the processor. If the system determines the moving object is not a human (e.g., was an animal or an inanimate object moving past the at least one camera), then the system can shut off or go back to a scanning for movement mode, optionally with a time delay for the non-human moving object to be removed from the frame of the at least one camera. If the system determines the moving object is a human, then the system can detect, via the at least one camera, if the face of said human is in a range of the directed light source used to provide the VL therapy. For example, the system can detect that the human sat down in from of the stand-alone appliance by detecting the facial height of the human. At step 708, the stand-alone appliance system can deliver VL therapy to at least one eye of the human.

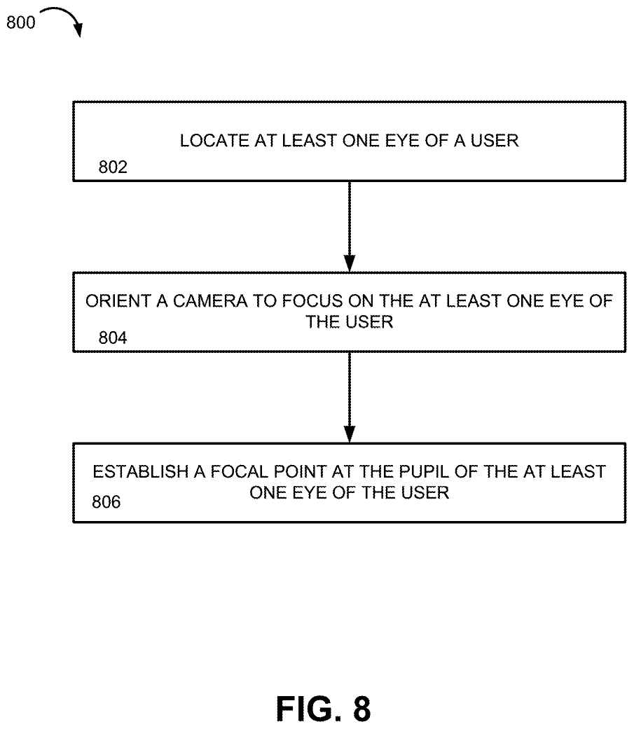

Referring now to FIG. 8, illustrated is a method 800 for delivering VL to at least one eye of a user. At step 802, the at least one eye of the user can be located by the processor via the at least one camera. In one instance both eyes of the user can be located at the same time or at different times. At step 804, the at least one camera can be oriented to focus on the at least one eye of the user via the at least one motor in communication with the processor. In one instance the at least one camera can be two cameras and each camera can be oriented to focus on one eye of the user. In another instance, the at least one camera can be one camera that is sequentially oriented to focus on each of the user for a given time period. At step 806, a focal point for the directed light source can be established at the pupil of the at least one eye of the user. In some instances, a focal point can be established at the pupil of both eyes of the user at the same time (e.g., if there is a second directed light source). In another instance a focal point can be established sequentially at a first eye and then a second eye of the user, each for a given time period.

Providing the directed therapeutic VL treatment to the at least one eye of the user via the directed light source of the stand-alone appliance system can further include the following steps. A distance between the directed light source and the at least one eye of the user can be measured at a given time. An intensity of the directed therapeutic light treatment can be adjusted based on the measured distance to the pupil of the at least one eye. The directed light source and the at least one camera can be oriented via the at least one motor to maintain the focal point for the directed light source at the pupil of the at least one eye of the user if the user moves. For example, if the user shifts their weight, fidgets, tilts their head, etc. The system can adjust a focal length of the directed light source based on the measured distance so that a pupil-sized light bead is centered on the pupil of the at least one eye of the user. In this way, the violet light is focused on the eye and does not illuminate the rest of the user (because violet light can be harmful to portions of the human body with too much exposure). These steps can be used for both eyes sequentially (each for a given time period one after the other) or simultaneously (if the stand alone appliance comprises at least two directed light sources). In the case of sequential application of the directed therapeutic light treatment then the system can apply the directed therapeutic light treatment to a first eye of the user for a first time period and then apply the directed therapeutic light treatment to a second eye of the user for a second time period after the first time period. The applications for each eye can be repeated a preprogrammed number of times until a full dose has been applied to both eyes and/or until the user can no longer be detected by the stand alone appliance (e.g., has walked away or moved out of frame).

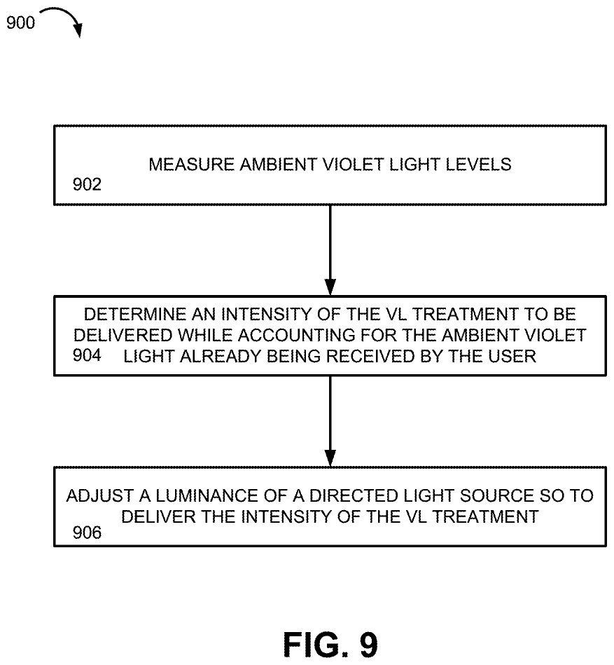

Referring now to FIG. 9, illustrated is a method 900 for regulating an amount of VL treatment delivered to a user. At step 902, ambient violet light levels near the stand-alone appliance system can be measured via an ambient light sensor in communication with the processor of the system. The ambient light sensor can be integrated with the stand-alone appliance or located at a second position near the stand-alone appliance and in wireless or wired communication with the stand-alone appliance. At step 904, an intensity of the VL treatment to be delivered to the user that accounts for the ambient violet light already being received by the user (e.g., based on the ambient violet light levels) can be determined. At step 906, a luminance of the directed light source can be adjusted so to deliver the intensity of the VL treatment that accounts for the ambient violet light. For example, if the ambient light source detects a high enough amount of violet light in the ambient light, then the directed therapeutic light treatment can have less intensity and/or be applied for a shorter time.

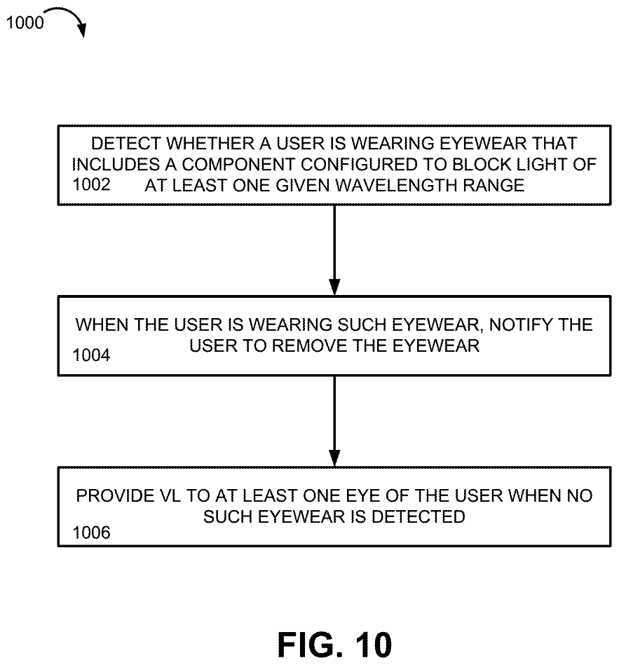

Referring now to FIG. 10, illustrated is a method 1000 for detecting eyewear of a user designed to block light. At step 1002, the system can detect whether the user is wearing eyewear (e.g., glasses, contact lenses, a monocle, an eye patch, etc.) that includes a component configured to block light of at least one given wavelength range (e.g., a wavelength range that can include violet light). At step 1006, when the user is wearing the eyewear, then the system can notify the user via a display and/or speaker associated with the system (e.g., via an auditory, visual, and/or haptic alert or message) to remove the eyewear that includes the component configured to block the light of the at least one given wavelength range via a display and/or speaker associated with the system. At step 1008, the directed therapeutic light treatment can be provided to the user when the system determines that no eyewear including the component configured to block the light of the at least one given wavelength range is detected.

From the above description, those skilled in the art will perceive improvements, changes, and modifications. Such improvements, changes and modifications are within the skill of one in the art and are intended to be covered by the appended claims.

The following is claimed:

1. A system comprising:
a fixture mounted on a platform;
at least one motor configured to move the platform;
a directed light source, coupled to a processor, configured to emit a light signal directed to an adjustable focal point;
a camera, coupled to the processor, configured to detect a presence of a user and provide an image of the user's face to the processor, wherein the processor signals the at least one motor to orient the platform to bring an eye into a center of a frame of the camera and to begin to adjust the adjustable focal point of the directed light source; and
a distance sensor, coupled to the processor, configured to estimate a distance between the fixture and the eye to ensure that an appropriate optical energy density is applied to the eye,
wherein at least one of the camera, the directed light source, and the distance sensor is embedded in the fixture.

2. The system of claim 1, wherein the directed light source comprises at least one of a violet light source and an infrared light source.

3. The system of claim 1, wherein the distance sensor comprises an ultrasound emitter, a light emitter, or an infrared emitter.

4. The system of claim 1, wherein the at least one motor is configured to move the platform in at least one degree of freedom.

5. The system of claim 1, wherein the at least one motor is configured to move the platform left/right and/or tilt the platform up/down.

6. The system of claim 1 further comprising:
a base attached to the platform and housing the processor and one or more of the at least one motor, the base further comprising:
at least a second camera, coupled to the processor, configured to detect the user; and
an ambient light detector, coupled to the processor, configured to detect an illuminance of ambient light to adjust an exposure time and an intensity of the light signal emitted by the directed light source.

7. The system of claim 6, wherein the base further comprises a non-transitory memory storing instructions coupled to the processor, the processor configured to execute the instructions to:
recognize the presence and identity of the user from a stored group of one or more users of the system; and
provide a directed therapeutic light treatment to the at least one eye of the user.

8. The system of claim 6, wherein the processor is configured to identify the user based on the image of the user's face detected by the camera and to determine a treatment length for the user.

9. The system of claim 8, wherein the treatment length is based on an amount of at least one type of light received by the user that day stored in the non-transitory memory.

10. The system of claim 6, wherein the at least the second camera is a wide angled camera.

11. The system of claim 8, the base further comprising at least one of: a power source and/or a charging port, a user interface, a display, a speaker, and circuitry.

12. A method comprising:
recognizing, by a system comprising a processor, the presence of a user via at least one camera in communication with the processor;
identifying, by the system, the user from a stored group of one or more users of the system; and
providing, by the system, a directed therapeutic light treatment to at least one eye of the user via a directed light source of the system, wherein at least one of the directed light source and the at least one camera are moveable by at least one motor in communication with the processor.

13. The method of claim 12, wherein the recognizing the presence of the user via the at least one camera in communication with the processor further comprises:
detecting, via the at the at least one camera, a moving object;
determining, by the system, that the moving object is a human; and
detecting, via the at least one camera, that a face of the human is in a range of the directed light source.

14. The method of claim 13, wherein the identifying the user from the group of one or more users of the system further comprises:
accessing, by the system, a database containing information about faces of the stored group of one or more users of the system;
performing, by the system, facial recognition of the face of the human compared to the information about faces of the stored group of one or more users of the system;
identifying, by the system, the human as the user of the system if the face of the human matches with information about one of the faces of the stored group of one or more users of the system

15. The method of claim 12, further comprising:
locating, by the processor via the at least one camera, the at least one eye of the user;
orienting, via the at least one motor in communication with the processor, the at least one camera to focus on the at least one eye of the user; and
establishing, by the system, a focal point for the directed light source at the pupil of the at least one eye of the user.

16. The method of claim 15, wherein the providing the directed therapeutic light treatment to the at least one eye of the user via the directed light source of the system further comprises:
measuring, by the system, a distance between the directed light source and the at least one eye of the user at a given time;
orienting, via the at least one motor, the directed light source and the at least one camera to maintain the focal point for the directed light source at the pupil of the at least one eye of the user if the user moves; and
adjusting, by the system, a focal length of the directed light source based on the measured distance so a pupil-sized light bead is centered on a pupil of the at least one eye of the user.

17. The method of claim 16, further comprising:
adjusting an intensity of the directed therapeutic light treatment based on the measured distance to the pupil of the at least one eye.

18. The method of claim 12, further comprising:
measuring, via an ambient light sensor in communication with the processor, ambient violet light levels near the system;
determining, by the system, an intensity of the directed therapeutic light treatment to account for the ambient violet light levels; and
adjusting, by the system, the intensity of the directed therapeutic light treatment by adjusting the luminance of the directed light source.

19. The method of claim 12, further comprising:
detecting, by the system, whether the user is wearing eyewear that includes a component configured to block light of at least one given wavelength range;
when the user is wearing the eyewear, notifying, by the system, the user to remove the eyewear that includes the component configured to block the light of the at least one given wavelength range via a display and/or speaker associated with the system; and
providing, by the system, the directed therapeutic light treatment to the at least one eye of the user when no eyewear including the component configured to block the light of the at least one given wavelength range is detected.

20. The method of claim 12, wherein providing the directed therapeutic light treatment to the at least one eye of the user via the directed light source of the system further comprises:
applying, by the system, the directed therapeutic light treatment to a first eye of the user for a first time period; and
applying, by the system, the directed therapeutic light treatment to a second eye of the user, for a second time period after the first time period, wherein the applications are repeated for a preprogrammed number of times.
